(19) United States
(12) Patent Application Publication
(10) Pub. No.: US 2015/0087662 A1
(43) Pub. Date: Mar. 26, 2015

(54) CYCLOBUTANE AND METHYLCYCLOBUTANE DERIVATIVES AS JANUS KINASE INHIBITORS

(71) Applicant: Incyte Corporation, Wilmington, DE (US)

(72) Inventors: Yun-Long Li, Chadds Ford, PA (US); James D. Rodgers, Landenberg, PA (US)

(21) Appl. No.: 14/499,916

(22) Filed: Sep. 29, 2014

Related U.S. Application Data
(63) Continuation of application No. 13/030,682, filed on Feb. 18, 2011, now abandoned.
(60) Provisional application No. 61/305,630, filed on Feb. 18, 2010.

(51) Int. Cl. C07D 487/04

(52) U.S. Cl. CPC C07D 487/04
USPC 514/265.1; 544/280

(57) ABSTRACT

The present invention relates to cyclobutane and methylcyclobutane derivatives, as well as their salts, compositions, and methods of use, which are Janus kinase (JAK) inhibitors useful in the treatment of JAK-associated diseases including, for example, inflammatory and autoimmune disorders, as well as cancer and myeloproliferative disorders.

### RELATED APPLICATION

This application claims priority to U.S. Provisional Application No. 61/305,630, filed Feb. 18, 2010, titled “CYCLOBUTANE AND METHYLCYCLOBUTANE DERIVATIVES AS JANUS KINASE INHIBITORS.” The contents of any patents, patent applications, and references cited throughout this specification are hereby incorporated by reference in their entireties.

### FIELD OF THE INVENTION

The present invention relates to cyclobutane and methylcyclobutane derivatives, as well as their salts, compositions, and methods of use. These compounds are Janus kinase (JAK) inhibitors useful in the treatment of JAK-associated diseases including, for example, inflammatory and autoimmune disorders, as well as cancer and myeloproliferative disorders.

### BACKGROUND OF THE INVENTION

Protein kinases (PKs) are a group of enzymes that regulate diverse, important biological processes including cell growth, survival and differentiation, organ formation and morphogenesis, neovascularization, tissue repair and regeneration, among others. Protein kinases exert their physiological functions through catalyzing the phosphorylation of proteins (or substrates) and thereby modulating the cellular activities of the substrates in various biological contexts. In addition to the functions in normal tissues/organs, many protein kinases also play more specialized roles in a host of human diseases, including cancer. A subset of protein kinases (also referred to as oncogenic protein kinases), when dysregulated, can cause tumor formation and growth, and further contribute to tumor maintenance and progression. Thus far, oncogenic protein kinases represent one of the largest and most attractive groups of protein targets for cancer intervention and drug development.

The Janus Kinase (JAK) family plays a role in the cytokine-dependent regulation of proliferation and function of cells involved in immune response. Currently, there are four known mammalian JAK family members: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2). The JAK proteins range in size from 120 to 140 kDa and comprise seven conserved JAK homology (JH) domains; one of these is a functional catalytic kinase domain, and another is a pseudokinase domain potentially serving a regulatory function and/or serving as a docking site for STATs.

Blocking signal transduction at the level of the JAK kinases holds promise for developing treatments for inflammatory diseases, autoimmune diseases, myeloproliferative diseases, and human cancers, to name a few Inhibition of the JAK kinases is also envisioned to have therapeutic benefits in patients suffering from skin immune disorders such as psoriasis, and skin sensitization.

Thus, new or improved agents that inhibit kinases such as Janus kinases are continually needed for developing new and more effective pharmaceuticals to treat cancer and other diseases. The compounds, salts, and compositions described herein are directed toward these needs and other ends.

### SUMMARY OF THE INVENTION

The present invention provides a compound which is 3-cyclobutyl-3-[4-(7H-pyrrolo[2,3-d]pyrimidin-4-yl)-1H-pyrazol-1-yl]propanenitrile, or a pharmaceutically acceptable salt thereof. In some embodiments, the aforementioned compound is the R or S enantiomer.

The present invention further provides a compound which is 3-(4-(7H-pyrrolo[2,3-d]pyrimidin-4-yl)-1H-pyrazol-1-yl)-3-(3-methylcyclobutyl)propanenitrile, or a pharmaceutically acceptable salt thereof. The present invention further includes the various stereoisomers of the aforementioned compound, including R and S enantiomers and cis and trans geometric isomers.

The present invention further provides a phosphoric acid salt of any of the cyclobutyl or methylcyclobutyl compounds described herein.

The present invention further provides a composition comprising a compound as described herein, or a pharmaceutically acceptable salt thereof, and at least one pharmaceutically acceptable carrier.

The present invention further provides methods of treating a JAK-associated disease or disorder in a patient comprising administering to the patient a therapeutically effective amount of a compound as described here, or a pharmaceutically acceptable salt thereof.

The present invention further provides the compounds described herein, or their pharmaceutically acceptable salts, for use in therapy.

The present invention further provides the use of the compounds described herein, or their pharmaceutically acceptable salts, for the preparation of a medicament for use in therapy.

Also provided herein is a method of treating an autoimmune disease in a patient comprising administering to said patient a therapeutically effective amount of a compound of the invention, or a pharmaceutically acceptable salt thereof. In one embodiment, the autoimmune disease is a skin disorder, multiple sclerosis, rheumatoid arthritis, psoriatic arthritis, juvenile arthritis, type I diabetes, lupus, inflammatory bowel disease, Crohn's disease, myasthenia gravis, immunoglobulin nephropathies, myocarditis, or autoimmune thyroid disorder. In another embodiment, the autoimmune disease is rheumatoid arthritis. In still another embodiment, the autoimmune disease is a skin disorder, such as atopic dermatitis, psoriasis, skin sensitization, skin irritation, skin rash, contact dermatitis or allergic contact sensitization.

In another aspect, provided herein is a method of treating cancer in a patient comprising administering to said patient a therapeutically effective amount of a compound of the invention, or a pharmaceutically acceptable salt thereof. In one embodiment, the cancer is a solid tumor. In another embodiment, the cancer is prostate cancer, renal cancer, hepatic cancer, breast cancer, lung cancer, thyroid cancer, Kaposi's sarcoma, Castleman's disease or pancreatic cancer. In still another embodiment, the cancer is lymphoma, leukemia, or multiple myeloma.

In still another aspect, provided herein is a method of treating a myeloproliferative disorder in a patient comprising administering to said patient a therapeutically effective amount of a compound of the invention, or a pharmaceutically acceptable salt thereof. In one embodiment, the myeloproliferative disorder (MPD) is polycythemia vera (PV), essential thrombocythemia (ET), primary myelofibrosis (PMF), myelofibrosis with myeloid metaplasia (MMM), chronic myelogenous leukemia (CML), chronic myelomonocytic leukemia (CMML), hypereosinophilic syndrome (HES), idiopathic myelofibrosis (IMF), systemic mast cell disease (SMCD), or post polycythemia vera/essential thrombocythemia myelofibrosis (Post-PV/ET MF).

In another aspect, provided herein is a method of treating an inflammatory disease in a patient comprising administering to said patient a therapeutically effective amount of a compound of the invention, or a pharmaceutically acceptable salt thereof.

In yet another aspect, provided herein is a method of treating organ transplant rejection in a patient comprising administering to said patient a therapeutically effective amount of a compound of the invention, or a pharmaceutically acceptable salt thereof.

In still another aspect, provided herein is a method of treating dry eye in a patient comprising administering to said patient a therapeutically effective amount of a compound of the invention, or a pharmaceutically acceptable salt thereof.

### DETAILED DESCRIPTION

The present invention provides, inter alia, the JAK-inhibiting compound:

3-cyclobutyl-3-[4-(7H-pyrrolo[2,3-d]pyrimidin-4-yl)-1H-pyrazol-1-yl]propanenitrile (Formula I), and its pharmaceutically acceptable salts.

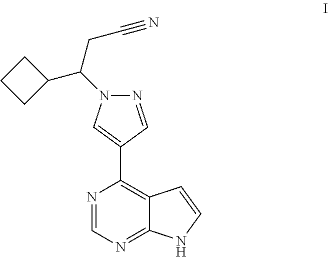 [SMILES: N#CCC(C1CCC1)n1cc(-c2ncnc3[nH]ccc23)cn1]

The present invention further provides the compounds (R)-3-cyclobutyl-3-[4-(7H-pyrrolo[2,3-d]pyrimidin-4-yl)-1H-pyrazol-1-yl]propanenitrile (Formula I-R) and (S)-3-cyclobutyl-3-[4-(7H-pyrrolo[2,3-d]pyrimidin-4-yl)-1H-pyrazol-1-yl]propanenitrile (Formula I-S), and their pharmaceutically acceptable salts.

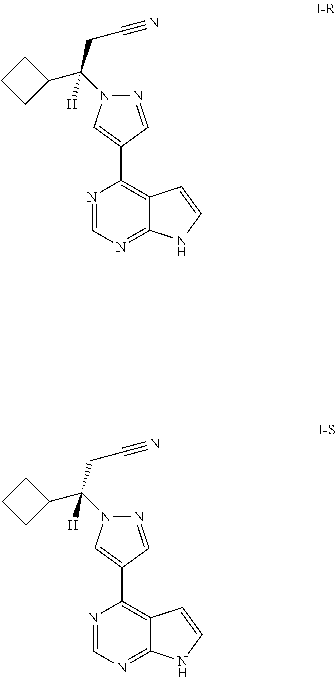

The present invention further provides the JAK-inhibiting compound 3-(4-(7H-pyrrolo[2,3-d]pyrimidin-4-yl)-1H-pyrazol-1-yl)-3-(3-methylcyclobutyl)propanenitrile (Formula II), and its pharmaceutically acceptable salts.

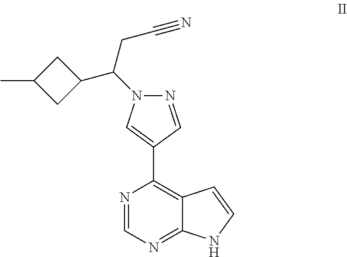 [SMILES: CC1CC(C(CC#N)n2cc(-c3ncnc4[nH]ccc34)cn2)C1]

The present invention further provides the cis and trans isomers of the compound of Formula II. These cis and trans isomers are:
3-(4-(7H-pyrrolo[2,3-d]pyrimidin-4-yl)-1H-pyrazol-1-yl)-3-((trans)-3-methylcyclobutyl)propanenitrile (Formula II-trans); and
3-(4-(7H-pyrrolo[2,3-d]pyrimidin-4-yl)-1H-pyrazol-1-yl)-3-((cis)-3-methylcyclobutyl)propanenitrile (Formula II-cis).

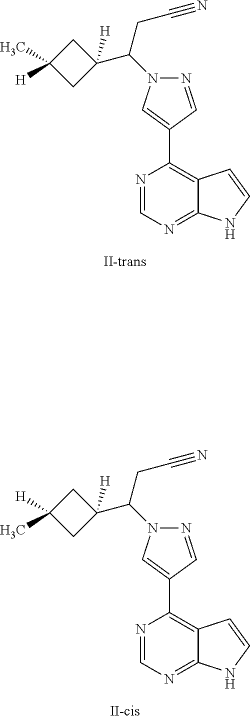 [SMILES: C[C@H]1C[C@H](C(CC#N)n2cc(-c3ncnc4[nH]ccc34)cn2)C1 ; C[C@H]1C[C@@H](C(CC#N)n2cc(-c3ncnc4[nH]ccc34)cn2)C1]

The present invention further provides the R and S enantiomers of the compound of Formula II. These R and S isomers are:
(3R)-3-(4-(7H-pyrrolo[2,3-d]pyrimidin-4-yl)-1H-pyrazol-1-yl)-3-((3-methylcyclobutyl)propanenitrile (Formula II-R); and
(3S)-3-(4-(7H-pyrrolo[2,3-d]pyrimidin-4-yl)-1H-pyrazol-1-yl)-3-(3-methylcyclobutyl)propanenitrile (Formula II-S).

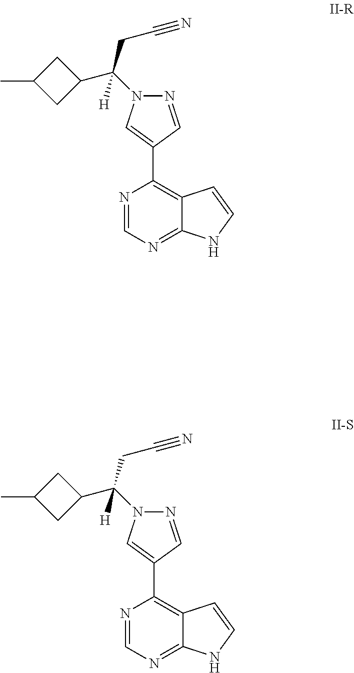

The present invention further provides the R/trans, R/cis, S/trans, and S/cis isomers of the compound of Formula II. These isomers are:
(3R)-3-(4-(7H-pyrrolo[2,3-d]pyrimidin-4-yl)-1H-pyrazol-1-yl)-3-((trans)-3-methylcyclobutyl)propanenitrile (Formula II-R/trans),
(3S)-3-(4-(7H-pyrrolo[2,3-d]pyrimidin-4-yl)-1H-pyrazol-1-yl)-3-((trans)-3-methylcyclobutyl)propanenitrile (Formula II-S/trans),
(3R)-3-(4-(7H-pyrrolo[2,3-d]pyrimidin-4-yl)-1H-pyrazol-1-yl)-3-((cis)-3-methylcyclobutyl)propanenitrile (Formula II-R/cis), are
(3S)-3-(4-(7H-pyrrolo[2,3-d]pyrimidin-4-yl)-1H-pyrazol-1-yl)-3-((cis)-3-methylcyclobutyl)propanenitrile (Formula II-S/cis).

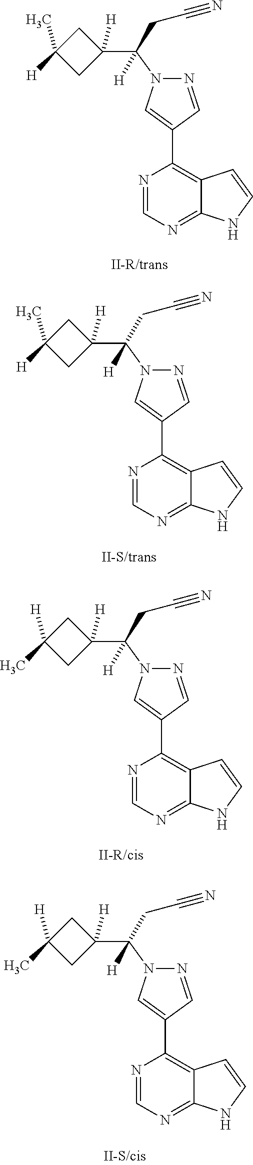

The compounds described above are referred to herein as “the compounds of the invention.” Here and elsewhere, where discrepancies exist between a compound's name and a compound's structure, the chemical structure will control.

The present invention further provides pharmaceutically acceptable salts of any of the aforementioned compounds. In some embodiments, the pharmaceutically acceptable salt is a phosphoric acid salt.

The compounds described herein are asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms are known in the art, such as by resolution of racemic mixtures or by stereoselective synthesis. Geometric isomers can also be present in the compounds described herein, and all such stable isomers are contemplated in the present invention. Cis and trans geometric isomers of the compounds of the present invention are described and may be isolated as a mixture of isomers or as substantially separated isomeric forms. Where a compound capable of stereoisomerism (e.g., optical and/or geometric isomerism) is designated in its structure or name without reference to specific R/S or cis/trans configurations, it is intended that all such isomers are contemplated. For example, Formulas I and II as depicted above are understood to be inclusive of both R and S isomers and cis and trans isomers to the extent the molecules allow for such isomerism.

Resolution of racemic mixtures, or separation of a mixture of optical and/or geometric isomers, can be carried out by any of numerous methods known in the art including chromatographic methods, (e.g., chiral column chromatography) or fractional rescrystallization.

Compounds of the invention may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole.

The compounds and salts of the present invention can be found together with other molecules, such as solvent and water molecules, to form hydrates and solvates.

Compounds and salts of the invention can also include all isotopes of atoms present within. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound or salt of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof.

The phrase “pharmaceutically acceptable” is employed herein to refer to those compounds, salts, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, the phrase “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Methods

Compounds and salts of the invention can inhibit activity of one or more Janus kinases (JAKs). JAKs to which the present compounds bind and/or inhibit include any member of the JAK family. The present compounds inhibit the activities of both JAK1 and JAK2.

Another aspect of the present invention pertains to methods of treating a JAK-associated disease or disorder in an individual (e.g., patient) by administering to the individual in need of such treatment a therapeutically effective amount or dose of a compound or salt of the present invention or a pharmaceutical composition thereof. A JAK-associated disease can include any disease, disorder or condition that is directly or indirectly linked to expression or activity of the JAK, including overexpression and/or abnormal activity levels. A JAK-associated disease can also include any disease, disorder or condition that can be prevented, ameliorated, or cured by modulating JAK activity.

Examples of JAK-associated diseases include diseases involving the immune system including, for example, organ transplant rejection (e.g., allograft rejection and graft versus host disease).

Further examples of JAK-associated diseases include autoimmune diseases such as multiple sclerosis, rheumatoid arthritis, juvenile arthritis, psoriatic arthritis, type I diabetes, lupus, psoriasis, inflammatory bowel disease, ulcerative colitis, Crohn's disease, myasthenia gravis, immunoglobulin nephropathies, autoimmune thyroid disorders, and the like. In some embodiments, the autoimmune disease is an autoimmune bullous skin disorder such as pemphigus vulgaris (PV) or bullous pemphigoid (BP).

Further examples of JAK-associated diseases include allergic conditions such as asthma, food allergies, atopic dermatitis and rhinitis. Further examples of JAK-associated diseases include viral diseases such as Epstein Barr Virus (EBV), Hepatitis B, Hepatitis C, HIV, HTLV 1, Varicella-Zoster Virus (VZV) and Human Papilloma Virus (HPV).

Further examples of JAK-associated diseases or conditions include skin disorders such as psoriasis (for example, psoriasis vulgaris), atopic dermatitis, skin rash, skin irritation, skin sensitization (e.g., contact dermatitis or allergic contact dermatitis). For example, certain substances including some pharmaceuticals when topically applied can cause skin sensitization. In some embodiments, co-administration or sequential administration of at least one JAK inhibitor of the invention together with the agent causing unwanted sensitization can be helpful in treating such unwanted sensitization or dermatitis. In some embodiments, the skin disorder is treated by topical administration of at least one JAK inhibitor of the invention.

In further embodiments, the JAK-associated disease is cancer including those characterized by solid tumors (e.g., prostate cancer, renal cancer, hepatic cancer, pancreatic cancer, gastric cancer, breast cancer, lung cancer, cancers of the head and neck, thyroid cancer, glioblastoma, Kaposi's sarcoma, Castleman's disease, melanoma etc.), hematological cancers (e.g., lymphoma, leukemia such as acute lymphoblastic leukemia, acute myelogenous leukemia (AML) or multiple myeloma), and skin cancer such as cutaneous T-cell lymphoma (CTCL) and cutaneous B-cell lymphoma. Example cutaneous T-cell lymphomas include Sezary syndrome and mycosis fungoides.

JAK-associated diseases can further include those characterized by expression of a mutant JAK2 such as those having at least one mutation in the pseudo-kinase domain (e.g., JAK2V617F).

JAK-associated diseases can further include myeloproliferative disorders (MPDs) such as polycythemia vera (PV), essential thrombocythemia (ET), myelofibrosis with myeloid metaplasia (MMM), chronic myelogenous leukemia (CML), chronic myelomonocytic leukemia (CMML), hypereosinophilic syndrome (HES), systemic mast cell disease (SMCD), and the like. In some embodiments, the myeloproliferative disorder is primary myelofibrosis (PMF) or post polycythemia vera/essential thrombocythemia myelofibrosis (Post-PV/ET MF).

Further JAK-associated diseases include inflammation and inflammatory diseases. Example inflammatory diseases include inflammatory diseases of the eye (e.g., iritis, uveitis, scleritis, conjunctivitis, or related disease), inflammatory diseases of the respiratory tract (e.g., the upper respiratory tract including the nose and sinuses such as rhinitis or sinusitis or the lower respiratory tract including bronchitis, chronic obstructive pulmonary disease, and the like), inflammatory myopathy such as myocarditis, and other inflammatory diseases.

The JAK inhibitors described herein can further be used to treat ischemia reperfusion injuries or a disease or condition related to an inflammatory ischemic event such as stroke or cardiac arrest. The JAK inhibitors described herein can further be used to treat anorexia, cachexia, or fatigue such as that resulting from or associated with cancer. The JAK inhibitors described herein can further be used to treat restenosis, sclerodermitis, or fibrosis. The JAK inhibitors described herein can further be used to treat conditions associated with hypoxia or astrogliosis such as, for example, diabetic retinopathy, cancer, or neurodegeneration. See, e.g., Dudley, A. C. et al. Biochem. J. 2005, 390(Pt 2):427-36 and Sriram, K. et al. J. Biol. Chem. 2004, 279(19):19936-47. Epub 2004 Mar. 2. The JAK inhibitors described herein can be used to treat Alzheimer's disease.

The JAK inhibitors described herein can further be used to treat other inflammatory diseases such as systemic inflammatory response syndrome (SIRS) and septic shock.

The JAK inhibitors described herein can further be used to treat gout and increased prostate size due to, e.g., benign prostatic hypertophy or benign prostatic hyperplasia.

The JAK inhibitors described herein, as well as other JAK inhibitors capable of influencing IL-6/STAT3 signaling, can further be used to treat inflammation-associated proliferative diseases. Inflammation has been shown to be linked to the development of certain types of cancers. For example, patients suffering from inflammatory bowel disease such as ulcerative colitis have been shown to have a much higher risk of developing colorectal cancer. These types of inflammation-linked cancers have been termed colitis-associated cancer (CAC). Several studies have shown that the IL-6/STAT3 signaling is involved in promoting CAC. For example, mice deficient in STAT3 intestinal epithelial cells had decreased tumor size and incidence in an animal model of CAC. Bromberg, et al., “Inflammation and cancer: IL-6 and STAT3 complete the link”, Cancer Cell, 15:79-80 (2009). Similar results were obtained with IL-6 deficient mice, which developed fewer and smaller adenomas than wild-type mice. Grivennikov, et al., “IL-6 and STAT3 are required for survival of intestinal epithelial cells and the development of colitis-associated cancer”, Cancer Cell, 15:103-111 (2009). See also, Bollrath, et al., “gp130-Mediated STAT3 activation in enterocytes regulatres cell survival and cell-cycle progression during colitis-associated tumorigenesis”, Cancer Cell, 15:91-102 (2009); and Kortylewski, et al., “Regulation of the IL-23 and IL-12 balance by Stat3 signaling in the tumor microenvironment”, Cancer Cell, 15:114-123 (2009).

Accordingly, in some embodiments, JAK inhibitors of the invention and those which influence IL-6/STAT3 signaling, can be used to treat inflammation-associated cancers. In some embodiments, the cancer is associated with inflammatory bowel disease. In some embodiments, the inflammatory bowel disease is ulcerative colitis. In some embodiments, the inflammatory bowel disease is Crohn's disease. In some embodiments, the inflammation-associated cancer is colitis-associated cancer. In some embodiments, the inflammation-associated cancer is colon cancer or colorectal cancer. In some embodiments, the cancer is gastric cancer, gastrointestinal carcinoid tumor, gastrointestinal stromal tumor (GIST), adenocarcinoma, small intestine cancer, or rectal cancer. In addition to the compounds provided herein, example JAK inhibitors that can be used in the treatment of inflammation-associated cancers include those described in US 2006/0106020; US 2006/0183906; US 2007/0149506; US 2007/0135461; US 2008/0188500; US 2008/0312258; US 2008/0312259; and U.S. Ser. No. 12/270,135.

JAK inhibitors can be tested in animal models for potential efficacy in treating inflammation-associated cancers. For example, CAC can be induced in treated (e.g., with JAK inhibitors) or untreated mice by the method summarized in Grivennikov, et al., “IL-6 and STAT3 are required for survival of intestinal epithelial cells and the development of colitis-associated cancer”, Cancer Cell, 15:103-111 (2009). Progression of the disease can be followed by measuring body weight and monitoring for signs of rectal bleeding and diarrhea. After sacrifice of the animals, portions of the distal colon are removed for analysis.

In some embodiments, the JAK inhibitors described herein can further be used to treat a dry eye disorder. As used herein, “dry eye disorder” is intended to encompass the disease states summarized in a recent official report of the Dry Eye Workshop (DEWS), which defined dry eye as “a multifactorial disease of the tears and ocular surface that results in symptoms of discomfort, visual disturbance, and tear film instability with potential damage to the ocular surface. It is accompanied by increased osmolarity of the tear film and inflammation of the ocular surface.” Lemp, “The Definition and Classification of Dry Eye Disease: Report of the Definition and Classification Subcommittee of the International Dry Eye WorkShop”, The Ocular Surface, 5(2), 75-92 April 2007, which is incorporated herein by reference in its entirety. Dry eye is also sometimes referred to as keratoconjunctivitis sicca. In some embodiments, the treatment of the dry eye disorder involves ameliorating a particular symptom of dry eye disorder, such as eye discomfort, visual disturbance, tear film instability, tear hyperosmolarity, and inflammation of the ocular surface. The use of JAK inhibitors for the treatment of dry eye is provided in U.S. Ser. No. 12/571,834, filed Oct. 1, 2009, which is incorporated herein by reference.

In a further aspect, the present invention provides a method of treating conjunctivitis, uveitis (including chronic uveitis), chorioditis, retinitis, cyclitis, sclieritis, episcleritis, or iritis; treating inflammation or pain related to corneal transplant, LASIK (laser assisted in situ keratomileusis), photorefractive keratectomy, or LASEK (laser assisted sub-epithelial keratomileusis); inhibiting loss of visual acuity related to corneal transplant, LASIK, photorefractive keratectomy, or LASEK; or inhibiting transplant rejection in a patient in need thereof, comprising administering to the patient a therapeutically effective amount of a compound of Formula I, or pharmaceutically acceptable salt or N-oxide thereof. In some embodiments, the compound, or pharmaceutically acceptable salt or N-oxide thereof, is administered preoperatively to a patient about to undergo a procedure selected from corneal transplant, LASIK, photorefractive keratectomy, and LASEK. In some embodiments, the compound, or pharmaceutically acceptable salt or N-oxide thereof, suppresses or lessens inflammation or pain during and after the procedure. In some embodiments, the compound, or pharmaceutically acceptable salt or N-oxide thereof, is administered about 1 day to about 2 days prior to the procedure. In some embodiments, the compound, or pharmaceutically acceptable salt or N-oxide thereof, is administered postoperatively to a patient who has undergone a procedure selected from corneal transplant, LASIK, photorefractive keratectomy, and LASEK. In some embodiments, inhibiting loss of visual acuity means lessening the loss of visual acuity. In some embodiments, the postoperative or preoperative treatment lessens the amount of scarring and fibrous deposits following the procedure. In some embodiments, inhibiting loss of visual acuity means that the patient retains visual acuity. In some embodiments, inhibiting transplant rejection means that the compound, or pharmaceutically acceptable salt or N-oxide thereof, is immunosuppressive, thereby preventing total rejection of the corneal transplant.

In one embodiment, provided herein is a method of treating cancer in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof. In another embodiment, provided herein is a method of treating myelofibrosis in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof. In another embodiment, provided herein is a method of treating rheumatoid arthritis (RA) in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof. In another embodiment, provided herein is a method of treating polycythemia vera (PV) in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof. In another embodiment, provided herein is a method of treating essential thrombocythemia (ET) in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof. In another embodiment, provided herein is a method of treating a solid tumor in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof. In another embodiment, provided herein is a method of treating psoriasis in a subject, comprising administering to the subject 3-cyclobutyl-3-[4-(7H-pyrrolo[2,3-d]pyrimidin-4-yl)-1H-pyrazol-1-yl]propanenitrile, an isomer thereof, or a pharmaceutically acceptable salt thereof.

In one embodiment, provided herein is a method of treating cancer in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof. In another embodiment, provided herein is a method of treating myelofibrosis in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof. In another embodiment, provided herein is a method of treating rheumatoid arthritis (RA) in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof. In another embodiment, provided herein is a method of treating polycythemia vera (PV) in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof. In another embodiment, provided herein is a method of treating essential thrombocythemia (ET) in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof. In another embodiment, provided herein is a method of treating a solid tumor in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof. In another embodiment, provided herein is a method of treating psoriasis in a subject, comprising administering to the subject 3-(4-(7H-pyrrolo[2,3-d]pyrimidin-4-yl)-1H-pyrazol-1-yl)-3-(3-methylcyclobutyl)propanenitrile, an isomer thereof, or a pharmaceutically acceptable salt thereof.

As used herein, the term “contacting” refers to the bringing together of indicated moieties in an in vitro system or an in vivo system. For example, “contacting” a JAK with a compound of the invention includes the administration of a compound of the present invention to an individual or patient, such as a human, having a JAK, as well as, for example, introducing a compound of the invention into a sample containing a cellular or purified preparation containing the JAK.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder; and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

The term “use” includes any one or more of the following embodiments of the invention, respectively: the use in the treatment of a disorder; the use for the manufacture of pharmaceutical compositions for use in the treatment of a disorder, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from diseases associated with the activity of JAK kinase.

### Combination Therapies

One or more additional pharmaceutical agents such as, for example, chemotherapeutics, anti-inflammatory agents, steroids, immunosuppressants, as well as Bcr-Abl, Flt-3, RAF and FAK kinase inhibitors such as, for example, those described in WO 2006/056399, or other therapeutic agents can be used in combination with the compounds or salts of the present invention for treatment of JAK-associated diseases, disorders or conditions. The one or more additional pharmaceutical agents can be administered to a patient simultaneously or sequentially.

Example chemotherapeutic include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include coriticosteroids such as dexamethasone or prednisone.

Example Bcr-Abl inhibitors include the compounds, and pharmaceutically acceptable salts thereof, of the genera and species disclosed in U.S. Pat. No. 5,521,184, WO 04/005281, and WO 2005/123719.

Example suitable Flt-3 inhibitors include compounds, and their pharmaceutically acceptable salts, as disclosed in WO 03/037347, WO 03/099771, and WO 04/046120.

Example suitable RAF inhibitors include compounds, and their pharmaceutically acceptable salts, as disclosed in WO 00/09495 and WO 05/028444.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402.

In some embodiments, one or more of the compounds of the invention can be used in combination with one or more other kinase inhibitors including imatinib, particularly for treating patients resistant to imatinib or other kinase inhibitors.

In some embodiments, one or more JAK inhibitors of the invention can be used in combination with a chemotherapeutic in the treatment of cancer and may potentially improve the treatment response as compared to the response to the chemotherapeutic agent alone, without exacerbation of its toxic effects. Examples of additional pharmaceutical agents used in the treatment of multiple myeloma, for example, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. Additive or synergistic effects are desirable outcomes of combining a JAK inhibitor of the present invention with an additional agent. Furthermore, resistance of multiple myeloma cells to agents such as dexamethasone may be reversible upon treatment with a JAK inhibitor of the present invention. The agents can be combined with the present compounds in a single or continuous dosage form, or the agents can be administered simultaneously or sequentially as separate dosage forms.

In some embodiments, a corticosteroid such as dexamethasone is administered to a patient in combination with at least one JAK inhibitor where the dexamethasone is administered intermittently as opposed to continuously.

In some further embodiments, combinations of one or more JAK inhibitors of the invention with other therapeutic agents can be administered to a patient prior to, during, and/or after a bone marrow transplant or stem cell transplant.

In some embodiments, at least one additional therapeutic agent can be used in connection with treatment of dry eye disorders and other disorders of the eye. In some embodiments, the additional therapeutic agent is fluocinolone acetonide (Retisert®), or rimexolone (AL-2178, Vexol, Alcon). In some embodiments, the additional therapeutic agent is cyclosporine (Restasis®). In some embodiments, the additional therapeutic agent is a corticosteroid. In some embodiments, the corticosteroid is triaminolone, dexamethasone, fluocinolone, cortisone, prednisolone, or flumetholone.

In some embodiments, the additional therapeutic agent is selected from Dehydrex™ (Holles Labs), Civamide (Opko), sodium hyaluonate (Vismed, Lantibio/TRB Chemedia), cyclosporine (ST-603, Sirion Therapeutics), ARG101(T) (testosterone, Argentis), AGR1012(P) (Argentis), ecabet sodium (Senju-Ista), gefarnate (Santen), 15-(s)-hydroxyeicosatetraenoic acid (15(S)-HETE), cevilemine, doxycline (ALTY-0501, Alacrity), minocycline, iDestrin™ (NP50301, Nascent Pharmaceuticals), cyclosporine A (Nova22007, Novagali), oxytetracycline (Duramycin, MOLI1901, Lantibio), CF101 (2S,3S,4R,5R)-3,4-dihydroxy-5-[6-[(3-iodophenyl)methylamino]purin-9-yl]-N-methyl-oxolane-2-carbamyl, Can-Fite Biopharma), voclosporin (LX212 or LX214, Lux Biosciences), ARG103 (Agentis), RX-10045 (synthetic resolvin analog, Resolvyx), DYN15 (Dyanmis Therapeutics), rivoglitazone (DE011, Daiichi Sanko), TB4 (RegeneRx), OPH-01 (Ophtalmis Monaco), PCS101 (Pericor Science), REV1-31 (Evolutec), Lacritin (Senju), rebamipide (Otsuka-Novartis), OT-551 (Othera), PAI-2 (University of Pennsylvania and Temple University), pilocarpine, tacrolimus, pimecrolimus (AMS981, Novartis), loteprednol etabonate, rituximab, diquafosol tetrasodium (INS365, Inspire), KLS-0611 (Kissei Pharmaceuticals), dehydroepiandrosterone, anakinra, efalizumab, mycophenolate sodium, etanercept (Embrel®), hydroxychloroquine, NGX267 (TorreyPines Therapeutics), or thalidomide.

In some embodiments, the additional therapeutic agent is an anti-angiogenic agent, cholinergic agonist, TRP-1 receptor modulator, a calcium channel blocker, a mucin secretagogue, MUC1 stimulant, a calcineurin inhibitor, a corticosteroid, a P2Y2 receptor agonist, a muscarinic receptor agonist, another JAK inhibitor, Bcr-Abl kinase inhibitor, Flt-3 kinase inhibitor, RAF kinase inhibitor, and FAK kinase inhibitor such as, for example, those described in WO 2006/056399. In some embodiments, the additional therapeutic agent is a tetracycline derivative (e.g., minocycline or doxycline).

In some embodiments, the additional therapeutic agent(s) are demulcent eye drops (also known as “artificial tears”), which include, but are not limited to, compositions containing polyvinylalcohol, hydroxypropyl methylcellulose, glycerin, polyethylene glycol (e.g. PEG400), or carboxymethyl cellulose. Artificial tears can help in the treatment dry eye by compensating for reduced moistening and lubricating capacity of the tear film. In some embodiments, the additional therapeutic agent is a mucolytic drug, such as N-acetyl-cysteine, which can interact with the mucoproteins and, therefore, to decrease the viscosity of the tear film.

In some embodiments, the additional therapeutic agent includes an antibiotic, antiviral, antifungal, anesthetic, anti-inflammatory agents including steroidal and non-steroidal anti-inflammatories, and anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; comolyn; lodoxamide; levocabastin; naphazoling; antazoline; pheniramimane; or azalide antibiotic.

### Pharmaceutical Formulations and Dosage Forms

When employed as pharmaceuticals, the compounds and salts of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

This invention also includes pharmaceutical compositions which contain, as the active ingredient, one or more of the compounds of the invention above in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, for example see International Patent Application No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

In some embodiments, the compound of the invention, or pharmaceutically acceptable salt thereof, is administered as an ophthalmic composition. Accordingly, in some embodiments, the methods comprise administration of the compound, or pharmaceutically acceptable salt thereof, and an ophthalmically acceptable carrier. In some embodiments, the ophthalmic composition is a liquid composition, semi-solid composition, insert, film, microparticles or nanooparticles. Ophthalmic compositions are described in detail in U.S. Ser. No. 12/571,834, filed Oct. 1, 2009, which is incorporated herein by reference.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Various modifications of the invention, in addition to those described herein, will be apparent to those skilled in the art from the description. Each reference cited in the present application is incorporated herein by reference in its entirety.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### (R or S)-3-Cyclobutyl-3-[4-(7H-pyrrolo[2,3-d]pyrimidin-4-yl)-1H-pyrazol-1-yl]propanenitrile

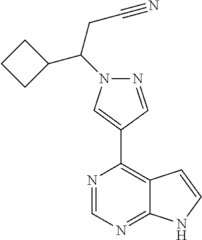 [SMILES: N#CCC(C1CCC1)n1cc(-c2ncnc3[nH]ccc23)cn1]

### Step 1. Cyclobutanecarboxaldehyde

A solution of dimethyl sulfoxide (34.6 mL, 0.488 mol) in methylene chloride (100 mL) was added to oxalyl chloride (20.6 mL, 0.244 mol) in methylene chloride (700 mL, 10 mol) at −78° C. After 10 min, cyclobutylmethanol (Aldrich, 17.5 g, 0.203 mol) in methylene chloride (100 mL) was added and the resultant mixture was stirred at −78° C. for 30 min. A solution of triethylamine (140 mL, 1.0 mol) in methylene chloride (100 mL) was then added and the mixture was stirred for 5 h with the temperature allowed to gradually warm up to room temperature (rt). After quenching with water, the mixture was separated. The organic layer was washed with water (×2), brine, dried over sodium sulfate, and filtered. The filtrate was distilled, collecting the 86-92° C. fraction to give the aldehyde (18.6 g, 54.4%).

### Step 2. 3-Cyclobutylacrylonitrile

To a solution of 1.00 M potassium tert-butoxide in tetrahydrofuran (116 mL, 0.116 mol) at 0° C. was added dropwise a solution of diethyl cyanomethylphosphonate (Aldrich, 19.7 mL, 0.122 mol) in tetrahydrofuran (200 mL). The reaction was warmed to rt and then cooled at 0° C. again. To the reaction mixture was a solution of cyclobutanecarboxaldehyde (see Step 1, 18.6 g, 0.110 mol) in tetrahydrofuran (100 mL). The reaction was allowed to warm up to room temperature (rt) and stirred at rt overnight. After quenching with water, the mixture was extracted with ether. The combined organic layers were washed with water, brine, dried and evaporated to dryness. The crude mixture was purified on silica gel, eluting with 0 to 40% EtOAc in hexane, to give the desired product (5.30 g, 44.7%). LCMS calculated for C₇H₁₀N(M+H)+: m/z=108.1. Found: 108.1.

### Step 3. (R)-3-Cyclobuyl-3-[4-(7-{[2-(trimethylsilyl)ethoxy]methyl}-7H-pyrrolo[2,3-d]pyrimidin-4-yl)-1H-pyrazol-1-yl]propanenitrile and (S)-3-Cyclobutyl-3-[4-(7-{[2-(trimethylsilyl)ethoxy]-methyl}-7H-pyrrolo[2,3-d]pyrimidin-4-yl)-1H-pyrazol-1-yl]propanenitrile

To a solution of 4-(1H-pyrazol-4-yl)-7-{[2-(trimethylsilyl)ethoxy]methyl}-7H-pyrrolo[2,3-d]pyrimidine (see U.S. Pub. No. US 2007/0135461, 15.6 g, 0.050 mol) in acetonitrile (124 mL, 2.37 mol) was added 3-cyclobutylacrylonitrile (5.30 g, 0.050 mol), followed by 1,8-diazabicyclo[5.4.0]undec-7-ene (3.70 mL, 0.025 mol). The resulting mixture was stirred at rt overnight, then evaporated to dryness. The mixture was purified on silica gel, eluting with 0 to 60% EtOAc in hexane, to give the desired product as a racemic mixture (16 g, 76%). LCMS calculated for C₂₂H₃₁N₆OSi(M+H)+: m/z=423.2. Found: 423.0. The racemic mixture was separated with chiral HPLC (Column: ChiralCel OJ-H, 30×250 mm, 5 μm; Mobile Phase: 30% Ethanol/70% Hexanes; Flow Rate: 24 mL/min) to give two enantiomers. On chiral analytical HPLC (Column: ChiralCel OJ-H, 4.6×250 mm, 5 μm; Mobile Phase: 30% ethanol/70% hexanes; Flow Rate: 0.8 mL/min): First peak retention time: 6.6 min; Second peak retention time: 8.1 min.

### Step 4. (R or S)-3-Cyclobuyl-3-[4-(7H-pyrrolo[2,3-d]pyrimidin-4-yl)-1H-pyrazol-1-yl]propanenitrile

In a 500 mL round bottom flask fitted with a stir bar, condenser, and nitrogen inlet was charged acetonitrile (55 mL), water (4.8 mL) and (R or S)-3-cyclobutyl-3-[4-(7-{[2-(trimethylsilyl)ethoxy]methyl}-7H-pyrrolo[2,3-d]pyrimidin-4-yl)-1H-pyrazol-1-yl]propanenitrile (second peak from chiral separation in Step 3, 2.8 g, 6.6 mmol). Lithium tetrafluoroborate (7.50 g, 0.078 mol) was added. The resultant mixture was warmed to reflux overnight, cooled to room temperature and charged with 3.00 M of ammonium hydroxide in water (9.78 mL) in portions over a period of 5 minutes adjusting pH to 9-10. After 30 min, the resulting mixture was purified by RP-HPLC (XBridge C18 30×100 mm column, with injection volume 5 mL (−50 mg/injection)), eluting with a gradient of acetonitrile/water containing 0.15% NH₄OH, at a flow rate of 60 mL/min) to give the desired product as a free base (1.51 g, 77.96%). LCMS calculated for C₁₆H₁₇N₆(M+H)+: m/z=293.2. Found: 293.1. ¹H NMR (300 MHz, CD₃OD) δ 8.65 (1H, s), 8.59 (1H, s), 8.34 (1H, s), 7.50 (1H, d, J=3.6 Hz), 6.94 (1H, d, J=3.6 Hz), 4.69 (1H, m), 3.07-2.97 (3H, m), 2.21 (1H, m), 1.97-1.84 (5H, m) ppm. ee 98.8%.

The other enantiomer can be prepared in the same manner starting with the compound corresponding to the first peak obtained from the chiral separation in Step 3.

### Example 2

### (3R or 3S)-3-(4-(7H-Pyrrolo[2,3-d]pyrimidin-4-yl)-1H-pyrazol-1-yl)-3-((trans)-3-methylcyclobutyl)propanenitrile phosphoric acid salt

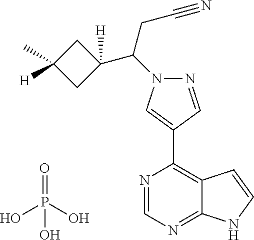 [SMILES: O=P(O)(O)O ; C[C@H]1C[C@H](C(CC#N)n2cc(-c3ncnc4[nH]ccc34)cn2)C1]

### Step 1. 3-Methylenecyclobutanecarboxylic acid

Into a round bottom flask equipped with a condenser was added 3-methylenecyclobutanecarbonitrile (BePharma, 10.0 g, 0.107 mol). To the flask was added a solution of potassium hydroxide (24.1 g, 0.365 mol) in ethanol (112 mL) and water (88 mL) and the mixture was heated at 100° C. After about 2 hours, ammonia evolution ceased and the solvent was evaporated to dryness under reduced pressure. The solids were dissolved in water (75 mL), cooled in an ice-bath, and acidified to pH of about 1 with concentrated hydrochloric acid. The resulting upper layer was extracted with dichloromethane twice. The organic layers were combined and dried over anhydrous magnesium sulfate. Removal of the organic solvents gave the desired crude product (11.8 g, 97.67%).

### Step 2. N-methoxy-N-methyl-3-methylenecyclobutanecarboxamide

To a mixture of 3-methylenecyclobutanecarboxylic acid (Step 1, 5.88 g, 52.4 mmol) in methylene chloride (100 mL) was added oxalyl chloride (Aldrich, 5.33 mL, 62.9 mmol), followed by a catalytic amount of dimethyl formamide (DMF). The reaction was stirred at rt for 2 h, then evaporated to dryness. The crude acid chloride was dissolved in methylene chloride (200 mL). To the resulting solution was added N,O-dimethylhydroxylamine hydrochloride ((Aldrich, 0.14 g, 62.9 mmol), followed by triethylamine (TEA) (21.9 mL, 0.157 mol), dropwsie, at 0° C. The reaction was stirred at rt overnight, and TEA HCl salt was filtered out. The filtrate was washed with 1 N HCl, then aq. sodium bicarbonate, brine, and dried over magnesium sulfate and evaporated to dryness. The crude amide (7.30 g, 89.7%) was used directly in next step. LCMS calculated for C₈H₁₄NO₂(M+H)+: m/z=156.1. Found: 156.3.

### Step 3. 3-Methylenecyclobutanecarbaldehyde

To a suspension of lithium tetrahydroaluminate (2.18 g, 57.5 mmol) in ether (200 mL) was added dropwise a solution of N-methoxy-N-methyl-3-methylenecyclobutanecarboxamide (Step 2, 7.14 g, 46.0 mmol) in tetrahydrofuran (75 mL) at −15° C. The reaction was stirred at 0 to −15° C. for 30 min, then quenched with aq. potassium hydrogen sulfate. The resulting mixture was extracted with ether. The combined organic layers were washed with brine, dried over magnesium sulfate, and evaporated. The crude product (6.70 g, 151.5%) was used directly in next step.

### Step 4. 3-(3-Methylenecyclobutyl)acrylonitrile

To a solution of 1.00 M of potassium tert-butoxide in tetrahydrofuran (48.3 mL, 48.3 mmol) at 0° C. was added dropwise a solution of diethyl cyanomethylphosphonate (Aldrich, 8.19 mL, 50.6 mmol) in tetrahydrofuran (80 mL). The reaction was warmed to rt and then cooled at 0° C. To the reaction mixture was added a solution of 3-methylenecyclobutanecarbaldehyde (Step 4, 4.42 g, 46.0 mmol) in tetrahydrofuran (40 mL). The reaction was allowed to warm to rt and then was stirred at rt overnight. After quenching with water, the mixture was extracted with ether. The combined organic layers were washed with water, brine, dried and evaporated to dryness. The crude mixture (5.90 g, 107.7%) was used directly in next step.

### Step 5. 3-(3-Methylenecyclobuyl)-3-[4-(7-{[2-(trimethylsilyl)ethoxy]methyl}-7H-pyrrolo[2,3-d]pyrimidin-4-yl)-1H-pyrazol-1-yl]propanenitrile

To a solution of 4-(1H-pyrazol-4-yl)-7-{[2-(trimethylsilyl)ethoxy]methyl}-7H-pyrrolo[2,3-d]pyrimidine (see U.S. Pub. No. US 2007/0135461, 7.25 g, 23.0 mmol) in acetonitrile (57.4 mL) was added crude 3-(3-methylenecyclobutyl)acrylonitrile (Step 4, 2.74 g, 23.0 mmol), followed by 1,8-diazabicyclo[5.4.0]undec-7-ene (3.44 mL, 23.0 mmol). The resulting mixture was stirred at rt over the weekend, then evaporated to dryness. The residue was purified on silica gel, eluting with 0 to 80% EtOAc in hexane, to give the desired product (6.0 g, 60.1%). LCMS calculated for C₂₃H₃₁N₆OSi(M+H)+: m/z=435.2. Found: 435.4.

### Step 6. (3R or 3S)-3-((trans)-3-Methylcyclobuyl)-3-(4-(7-((2-(trimethylsilyl)ethoxy)methyl)-7H-pyrrolo[2,3-d]pyrimidin-4-yl)-1H-pyrazol-1-yl)propanenitrile

A mixture of 3-(3-methylenecyclobutyl)-3-[4-(7-{[2-(trimethylsilyl)ethoxy]methyl}-7H-pyrrolo[2,3-d]pyrimidin-4-yl)-1H-pyrazol-1-yl]propanenitrile (Step 5, 4.0 g, 9.2 mol) in 100 mL of methanol was hydrogenated in the presence of 0.6 g of 10% Pd/C, under balloon pressure of hydrogen, for 1 h. After filtering off the catalyst, the filtrate was evaporated to dryness and purified on silica gel, eluting with 0 to 100% EtOAc in hexane, to give the desired product as a mixture of trans- and cis-isomers. LCMS calculated for C₂₃H₃₃N₆OSi(M+H)+: m/z=437.3. Found: 437.4. The product was subjected to purification on chiral HPLC column twice. The first HPLC separation (Column: ChiralCel OD-H, 30×250 mm, 5 μm; Mobile Phase: 15% ethanol/85% hexanes; Flow Rate: 28 mL/min) gave two fractions, A and B. Fraction A was a cis/trans mixture of one enantiomer. Retention time: 10.51 min. Fraction B was a cis/trans mixture of the other enantiomer, which showed two inseparable peaks with retention times 13.05 min and 13.92 min. The first fraction (A) was subjected to further chiral HPLC separation (Column: ChiralPak IA, 20×250 mm, 5 μm; Mobile Phase: 10% ethanol/90% hexanes; Flow Rate: 15 mL/min) to give two peaks, A1 and A2, one peak corresponding to cis and the other to trans. According to chiral analytical HPLC (Column: ChiralPak IA, 4.6×250 mm, 5 μm; Mobile Phase: 15% ethanol/85% hexanes; Flow Rate: 1.0 mL/min): first peak (A1) retention time: 11.79 min; second peak (A2) retention time: 12.78 min. The second fraction (B) was subjected to chiral HPLC separation (Column: ChiralPak IA, 20×250 mm, 5 μm; Mobile Phase: 15% ethanol/85% hexanes; Flow Rate: 15 mL/min) to give two peaks, B1 and B2 (each peak 800 mg, 19.9%). B1 was later shown by nOe to be the cis-isomer and B2 was shown to be the trans-isomer of the other enantiomer. According to chiral analytical HPLC (Column: ChiralPak IA, 4.6×250 mm, 5 μm; Mobile Phase: 15% ethanol/85% hexanes; Flow Rate: 1.0 mL/min): first peak (B1) retention time: 12.48 min and second peak (B2) retention time: 14.16 min.

### Step 7. (3R or 3S)-3-(4-(7H-Pyrrolo[2,3-d]pyrimidin-4-yl)-1H-pyrazol-1-yl)-3-((trans)-3-methylcyclobutyl)propanenitrile

In a 500 mL round bottom flask fitted with stir bar, condenser and nitrogen inlet was charged acetonitrile (9.69 mL), water (0.84 mL) and 3-((trans)-3-methylcyclobutyl)-3-[4-(7-{[2-(trimethylsilyl)ethoxy]methyl}-7H-pyrrolo[2,3-d]pyrimidin-4-yl)-1H-pyrazol-1-yl]propanenitrile (0.60 g, 1.4 mol) (B2 from chiral separation in previous step (i.e., peak 2 of second fraction)). Lithium tetrafluoroborate (1.31 g, 13.7 mmol) was added. The mixture was warmed to reflux overnight, then charged with 7.2 M of ammonium hydroxide in water (0.71 mL, 5.1 mmol) in portions over a period of 5 minutes at room temperature adjusting pH to 9-10. The reaction was stirred for 2 h at room temperature. Solid was removed by filtration and the filtrate was purified on RP-HPLC ((XBridge C18 30×100 mm column, with injection volume 5 mL (−50 mg/injection), eluting with a gradient of acetonitrile/water containing 0.15% NH₄OH, at flow rate 60 mL/min) to yield the desired product as free base. LCMS calculated for C₁₇H₁₉N₆(M+H)+: m/z=307.2. Found: 307.4. ¹H NMR (500 MHz, DMSO-d₆) δ 12.08 (1H, s), 8.78 (1H, s), 8.68 (1H, s), 8.36 (1H, s), 7.59 (1H, d, J=3.0 Hz), 6.99 (1H, d, J=3.0 Hz), 4.78 (1H, m), 3.12 (2H, m), 2.88 (1H, m), 2.30 (1H, m), 2.06 (1H, m), 1.88 (1H, m), 1.74 (1H, m), 1.44 (1H, m), 1.08 (3H, d, J=7.0 Hz) ppm. ee 93.3%.

The other enantiomer can be prepared by the same method starting with the compound corresponding to fraction A from Step 6.

### Step 8. (3R or 3S)-3-(4-(7H-Pyrrolo[2,3-d]pyrimidin-4-yl)-1H-pyrazol-1-yl)-3-((trans)-3-methylcyclobutyl)propanenitrile phosphoric acid salt

To a solution of (3R or 3S)-3-((trans)-3-methylcyclobutyl)-3-[4-(7H-pyrrolo[2,3-d]pyrimidin-4-yl)-1H-pyrazol-1-yl]propanenitrile (Step 7, 0.275 g, 0.898 mmol) in isopropyl alcohol (5.83 mL) was added phosphoric acid (96.8 mg, 0.987 mmol) in 1.0 mL isopropanol at 60° C. After stirring for 1 h, the mixture was allowed to cool to rt. The precipitate was filtered off and air dried, then rinsed with ethyl ether and air dried further to give the desired phosphate product (330 mg, 90.9%).

### Example 3

### (3R or 3S)-3-(4-(7H-Pyrrolo[2,3-d]pyrimidin-4-yl)-1H-pyrazol-1-yl)-3-((cis)-3-methylcyclobutyl)-propanenitrile phosphoric acid salt

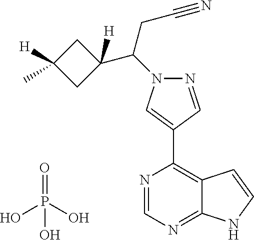 [SMILES: O=P(O)(O)O ; C[C@H]1C[C@@H](C(CC#N)n2cc(-c3ncnc4[nH]ccc34)cn2)C1]

### Step 1. (3R or 3S)-3-((cis)-3-Methylcyclobutyl)-3-(4-(7-((2-(trimethylsilyl)ethoxy)methyl)-7H-pyrrolo[2,3-d]pyrimidin-4-yl)-1H-pyrazol-1-yl)propanenitrile

In a 500 mL round bottom flask fitted with a stir bar, condenser, and nitrogen inlet was charged acetonitrile (8.1 mL), water (0.70 mL) and (3R or 3S)-3-((cis)-3-methylcyclobutyl)-3-[4-(7-{[2-(trimethylsilyl)ethoxy]methyl}-7H-pyrrolo[2,3-d]pyrimidin-4-yl)-1H-pyrazol-1-yl]propanenitrile (0.50 g, 1.1 mmol) (B1 from chiral separation described in Example 2, Step 6 (i.e., peak 1 of second fraction)). Lithium tetrafluoroborate (1.10 g, 11.4 mmol) was added. The solution was warmed to reflux overnight. Then a solution of ammonium hydroxide in water (7.2 M, 0.59 mL, 4.3 mmol) was charged to the solution in portions over a period of 5 minutes at room temperature adjusting pH to 9-10. The reaction was stirred for 2 h at room temperature. Solid was removed by filtration and the filtrate was purified on RP-HPLC (XBridge C18 30×100 mm column, with injection volume 5 mL (˜50 mg/injection), eluting with a gradient of acetonitrile/water containing 0.15% NH₄OH, at flow rate 60 mL/min) to give the desired product. LCMS calculated for C₁₇H₁₉N₆(M+H)+: m/z=307.2. Found: 307.4. ¹H NMR (500 MHz, DMSO-d₆) δ 12.08 (1H, s), 8.75 (1H, s), 8.68 (1H, s), 8.36 (1H, s), 7.59 (1H, d, J=3.0 Hz), 6.99 (1H, d, J=3.0 Hz), 4.66 (1H, m), 3.11 (2H, m), 2.66 (1H, m), 2.20 (2H, m), 1.88 (1H, m), 1.42 (2H, m), 0.97 (3H, d, J=6.0 Hz) ppm. ee 99.8%.

The other enantiomer can be prepared by the same method starting with the compound corresponding to fraction A from Example 2, Step 6.

### Step 2. (3R or 3S)-3-(4-(7H-pyrrolo[2,3-d]pyrimidin-4-yl)-1H-pyrazol-1-yl)-3-((cis)-3-methylcyclobutyl)propanenitrile phosphoric acid salt

To a solution of (3R or 3S)-3-((cis)-3-methylcyclobutyl)-3-[4-(7H-pyrrolo[2,3-d]pyrimidin-4-yl)-1H-pyrazol-1-yl]propanenitrile (Step 1, 0.23 g, 0.751 mmol) in isopropyl alcohol (4.87 mL) was added phosphoric acid (80.9 mg, 0.83 mmol) in 1.0 mL isopropanol at 60° C. The mixture was stirred for 2 h, then allowed to cool to rt. The precipitate was filtered off and air dried, then rinsed with ethyl ether and air dried further to give the desired phosphate product (300 mg, 98.8%). ¹H NMR (400 MHz, DMSO-d₆) δ 12.08 (1H, s), 8.75 (1H, s), 8.65 (1H, s), 8.34 (1H, s), 7.58 (1H, d, J=2.4 Hz), 6.97 (1H, d, J=2.4 Hz), 4.63 (1H, m), 3.09 (2H, m), 2.64 (1H, m), 2.18 (2H, m), 1.86 (1H, m), 1.40 (2H, m), 0.96 (3H, d, J=6.4 Hz) ppm.

### Example A

### In Vitro JAK Kinase Assay

Compounds herein were tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) with an N-terminal His tag were expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC₅₀s of compounds were measured for each kinase in the reactions that contain the enzyme, ATP and 500 nM peptide in 50 mM Tris (pH 7.8) buffer with 100 mM NaCl, 5 mM DTT, and 0.1 mg/mL (0.01%) BSA. The ATP concentration in the reactions was 90 μM for JAK1, 30 μM for JAK2 and 3 μM for JAK3. Reactions were carried out at room temperature for 1 hr and then stopped with 20 μL 45 mM EDTA, 300 nM SA-APC, 6 nM Eu-Py20 in assay buffer (Perkin Elmer, Boston, Mass.). Binding to the Europium labeled antibody took place for 40 minutes and HTRF signal was measured on a Fusion plate reader (Perkin Elmer, Boston, Mass.). The compounds of Examples 1, 2 and 3 were found to have IC₅₀ values less than 2 nM for JAK1 and less than 1 nM for JAK2.

### Example B

### Cellular Assays

One or more compounds herein were tested for inhibitory activity of JAK targets according to at least one of the following cellular assays.

Cancer cell lines dependent on cytokines and hence JAK/STAT signal transduction, for growth, were plated at 6000 cells per well (96 well plate format) in RPMI 1640, 10% FBS, and 1 nG/mL of appropriate cytokine Compounds were added to the cells in DMSO/media (final concentration 0.2% DMSO) and incubated for 72 hours at 37° C., 5% CO₂. The effect of compound on cell viability was assessed using the CellTiter-Glo Luminescent Cell Viability Assay (Promega) followed by TopCount (Perkin Elmer, Boston, Mass.) quantitation. Potential off-target effects of compounds were measured in parallel using a non-JAK driven cell line with the same assay readout. All experiments were performed in duplicate.

The above cell lines can also be used to examine the effects of compounds on phosphorylation of JAK kinases or potential downstream substrates such as STAT proteins, Akt, Shp2, or Erk. These experiments can be performed following an overnight cytokine starvation, followed by a brief preincubation with compound (2 hours or less) and cytokine stimulation of approximately 1 hour or less. Proteins are then extracted from cells and analyzed by techniques familiar to those schooled in the art including Western blotting or ELISAs using antibodies that can differentiate between phosphorylated and total protein. These experiments can utilize normal or cancer cells to investigate the activity of compounds on tumor cell survival biology or on mediators of inflammatory disease. For example, with regards to the latter, cytokines such as IL-6, IL-12, IL-23, or IFN can be used to stimulate JAK activation resulting in phosphorylation of STAT protein(s) and potentially in transcriptional profiles (assessed by array or qPCR technology) or production and/or secretion of proteins, such as IL-17. The ability of compounds to inhibit these cytokine mediated effects can be measured using techniques common to those schooled in the art.

Compounds herein can also be tested in cellular models designed to evaluate their potency and activity against mutant JAKs, for example, the JAK2V617F mutation found in myeloid proliferative disorders. These experiments often utilize cytokine dependent cells of hematological lineage (e.g. BaF/3) into which the wild-type or mutant JAK kinases are ectopically expressed (James, C., et al. Nature 434:1144-1148; Staerk, J., et al. JBC 280:41893-41899). Endpoints include the effects of compounds on cell survival, proliferation, and phosphorylated JAK, STAT, Akt, or Erk proteins.

Certain compounds herein have been or can be evaluated for their activity inhibiting T-cell proliferation. Such as assay can be considered a second cytokine (i.e. JAK) driven proliferation assay and also a simplistic assay of immune suppression or inhibition of immune activation. The following is a brief outline of how such experiments can be performed. Peripheral blood mononuclear cells (PBMCs) are prepared from human whole blood samples using Ficoll Hypaque separation method and T-cells (fraction 2000) can be obtained from PBMCs by elutriation. Freshly isolated human T-cells can be maintained in culture medium (RPMI 1640 supplemented withl0% fetal bovine serum, 100 U/ml penicillin, 100 μg/ml streptomycin) at a density of 2×10⁶ cells/ml at 37° C. for up to 2 days. For IL-2 stimulated cell proliferation analysis, T-cells are first treated with Phytohemagglutinin (PHA) at a final concentration of 10 μg/mL for 72 h. After washing once with PBS, 6000 cells/well are plated in 96-well plates and treated with compounds at different concentrations in the culture medium in the presence of 100 U/mL human IL-2 (ProSpec-Tany TechnoGene; Rehovot, Israel). The plates are incubated at 37° C. for 72 h and the proliferation index is assessed using CellTiter-Glo Luminescent reagents following the manufactory suggested protocol (Promega; Madison, Wis.).

### Example C

### In Vivo Anti-Tumor Efficacy

Compounds herein can be evaluated in human tumor xenograft models in immune compromised mice. For example, a tumorigenic variant of the INA-6 plasmacytoma cell line can be used to inoculate SCID mice subcutaneously (Burger, R., et al. Hematol J. 2:42-53, 2001). Tumor bearing animals can then be randomized into drug or vehicle treatment groups and different doses of compounds can be administered by any number of the usual routes including oral, i.p., or continuous infusion using implantable pumps. Tumor growth is followed over time using calipers. Further, tumor samples can be harvested at any time after the initiation of treatment for analysis as described above (Example B) to evaluate compound effects on JAK activity and downstream signaling pathways. In addition, selectivity of the compound(s) can be assessed using xenograft tumor models that are driven by other know kinases (e.g. Bcr-Abl) such as the K562 tumor model.

### Example D

### Murine Skin Contact Delayed Hypersensitivity Response Test

Compounds herein can also be tested for their efficacies (of inhibiting JAK targets) in the T-cell driven murine delayed hypersensitivity test model. The murine skin contact delayed-type hypersensitivity (DTH) response is considered to be a valid model of clinical contact dermatitis, and other T-lymphocyte mediated immune disorders of the skin, such as psoriasis (Immunol Today. 1998 January; 19(1):37-44). Murine DTH shares multiple characteristics with psoriasis, including the immune infiltrate, the accompanying increase in inflammatory cytokines, and keratinocyte hyperproliferation. Furthermore, many classes of agents that are efficacious in treating psoriasis in the clinic are also effective inhibitors of the DTH response in mice (Agents Actions. 1993 January; 38(1-2):116-21).

On Day 0 and 1, Balb/c mice are sensitized with a topical application, to their shaved abdomen with the antigen 2,4,dinitro-fluorobenzene (DNFB). On day 5, ears are measured for thickness using an engineer's micrometer. This measurement is recorded and used as a baseline. Both of the animals' ears are then challenged by a topical application of DNFB in a total of 20 μL (10 μL on the internal pinna and 10 μL on the external pinna) at a concentration of 0.2%. Twenty-four to seventy-two hours after the challenge, ears are measured again. Treatment with the test compounds was given throughout the sensitization and challenge phases (day −1 to day 7) or prior to and throughout the challenge phase (usually afternoon of day 4 to day 7). Treatment of the test compounds (in different concentration) was administered either systemically or topically (topical application of the treatment to the ears). Efficacies of the test compounds are indicated by a reduction in ear swelling comparing to the situation without the treatment. Compounds causing a reduction of 20% or more were considered efficacious. In some experiments, the mice are challenged but not sensitized (negative control).

The inhibitive effect (inhibiting activation of the JAK-STAT pathways) of the test compounds can be confirmed by immunohistochemical analysis. Activation of the JAK-STAT pathway(s) results in the formation and translocation of functional transcription factors. Further, the influx of immune cells and the increased proliferation of keratinocytes should also provide unique expression profile changes in the ear that can be investigated and quantified. Formalin fixed and paraffin embedded ear sections (harvested after the challenge phase in the DTH model) are subjected to immunohistochemical analysis using an antibody that specifically interacts with phosphorylated STAT3 (clone 58E12, Cell Signaling Technologies). The mouse ears are treated with test compounds, vehicle, or dexamethasone (a clinically efficacious treatment for psoriasis), or without any treatment, in the DTH model for comparisons. Test compounds and the dexamethasone can produce similar transcriptional changes both qualitatively and quantitatively, and both the test compounds and dexamethasone can reduce the number of infiltrating cells. Both systemically and topical administration of the test compounds can produce inhibitive effects, i.e., reduction in the number of infiltrating cells and inhibition of the transcriptional changes.

### Example E

### In Vivo Anti-Inflammatory Activity

Compounds herein can be evaluated in rodent or non-rodent models designed to replicate a single or complex inflammation response. For instance, rodent models of arthritis can be used to evaluate the therapeutic potential of compounds dosed preventatively or therapeutically. These models include but are not limited to mouse or rat collagen-induced arthritis, rat adjuvant-induced arthritis, and collagen antibody-induced arthritis. Autoimmune diseases including, but not limited to, multiple sclerosis, type I-diabetes mellitus, uveoretinitis, thyroditis, myasthenia gravis, immunoglobulin nephropathies, myocarditis, airway sensitization (asthma), lupus, or colitis may also be used to evaluate the therapeutic potential of compounds herein. These models are well established in the research community and are familiar to those schooled in the art (Current Protocols in Immunology, Vol 3., Coligan, J. E. et al, Wiley Press.; Methods in Molecular Biology: Vol. 225, Inflammation Protocols., Winyard, P. G. and Willoughby, D. A., Humana Press, 2003.).

### Example F

### Animal Models for the Treatment of Dry Eye, Uveitis, and Conjunctivitis

Compounds may be evaluated in one or more preclinical models of dry eye known to those schooled in the art including, but not limited to, the rabbit concanavalin A (ConA) lacrimal gland model, the scopolamine mouse model (subcutaneous or transdermal), the Botulinumn mouse lacrimal gland model, or any of a number of spontaneous rodent auto-immune models that result in ocular gland dysfunction (e.g. NOD-SCID, MRL/lpr, or NZB/NZW) (Barabino et al., Experimental Eye Research 2004, 79, 613-621 and Schrader et al., Developmental Opthalmology, Karger 2008, 41, 298-312, each of which is incorporated herein by reference in its entirety). Endpoints in these models may include histopathology of the ocular glands and eye (cornea, etc.) and possibly the classic Schirmer test or modified versions thereof (Barabino et al.) which measure tear production. Activity may be assessed by dosing via multiple routes of administration (e.g. systemic or topical) which may begin prior to or after measurable disease exists.

Compounds may be evaluated in one or more preclinical models of uveitis known to those schooled in the art. These include, but are not limited to, models of experimental autoimmune uveitis (EAU) and endotoxin induced uveitis (EIU). EAU experiements may be performed in the rabbit, rat, or mouse and may involve passive or activate immunization. For instance, any of a number or retinal antigens may be used to sensitize animals to a relevant immunogen after which animals may be challenged ocuarly with the same antigen. The EIU model is more acute and involves local or systemic administration of lipopolysaccaride at sublethal doses. Endpoints for both the EIU and EAU models may include fundoscopic exam, histopathology amongst others. These models are reviewed by Smith et al. (Immunology and Cell Biology 1998, 76, 497-512, which is incorporated herein by reference in its entirety). Activity is assessed by dosing via multiple routes of administration (e.g. systemic or topical) which may begin prior to or after measurable disease exists. Some models listed above may also develop scleritis/episcleritis, chorioditis, cyclitis, or iritis and are therefore useful in investigating the potential activity of compounds for the therapeutic treatment of these diseases.

Compounds may also be evaluated in one or more preclinical models of conjunctivitis known those schooled in the art. These include, but are not limited to, rodent models utilizing guinea-pig, rat, or mouse. The guinea-pig models include those utilizing active or passive immunization and/or immune challenge protocols with antigens such as ovalbumin or ragweed (reviewed in Groneberg, D. A., et al., Allergy 2003, 58, 1101-1113, which is incorporated herein by reference in its entirety). Rat and mouse models are similar in general design to those in the guinea-pig (also reviewed by Groneberg). Activity may be assessed by dosing via multiple routes of administration (e.g. systemic or topical) which may begin prior to or after measurable disease exists. Endpoints for such studies may include, for example, histological, immunological, biochemical, or molecular analysis of ocular tissues such as the conjunctiva.

Various modifications of the invention, in addition to those described herein, will be apparent to those skilled in the art from the above description. Each reference cited in the present application is incorporated herein by reference in its entirety.

1. A compound which is 3-cyclobutyl-3-[4-(7H-pyrrolo[2,3-d]pyrimidin-4-yl)-1H-pyrazol-1-yl]propanenitrile, or a pharmaceutically acceptable salt thereof.

2. The compound of claim 1 which is (R)-3-cyclobutyl-3-[4-(7H-pyrrolo[2,3-d]pyrimidin-4-yl)-1H-pyrazol-1-yl]propanenitrile, or a pharmaceutically acceptable salt thereof.

3. The compound of claim 1 which is (S)-3-cyclobutyl-3-[4-(7H-pyrrolo[2,3-d]pyrimidin-4-yl)-1H-pyrazol-1-yl]propanenitrile, or a pharmaceutically acceptable salt thereof.

4. A compound which is 3-(4-(7H-pyrrolo[2,3-d]pyrimidin-4-yl)-1H-pyrazol-1-yl)-3-(3-methylcyclobutyl)propanenitrile, or a pharmaceutically acceptable salt thereof.

5. The compound of claim 4 which is 3-(4-(7H-pyrrolo[2,3-d]pyrimidin-4-yl)-1H-pyrazol-1-yl)-3-((trans)-3-methylcyclobutyl)propanenitrile, or a pharmaceutically acceptable salt thereof.

6. The compound of claim 5 which is (3R)-3-(4-(7H-pyrrolo[2,3-d]pyrimidin-4-yl)-1H-pyrazol-1-yl)-3-((trans)-3-methylcyclobutyl)propanenitrile, or a pharmaceutically acceptable salt thereof.

7. The compound of claim 5 which is (3S)-3-(4-(7H-pyrrolo[2,3-d]pyrimidin-4-yl)-1H-pyrazol-1-yl)-3-((trans)-3-methylcyclobutyl)propanenitrile, or a pharmaceutically acceptable salt thereof.

8. The compound of claim 4 which is 3-(4-(7H-pyrrolo[2,3-d]pyrimidin-4-yl)-1H-pyrazol-1-yl)-3-((cis)-3-methylcyclobutyl)propanenitrile, or a pharmaceutically acceptable salt thereof.

9. The compound of claim 8 which is (3R)-3-(4-(7H-pyrrolo[2,3-d]pyrimidin-4-yl)-1H-pyrazol-1-yl)-3-((cis)-3-methylcyclobutyl)propanenitrile, or a pharmaceutically acceptable salt thereof.

10. The compound of claim 8 which is (3S)-3-(4-(7H-pyrrolo[2,3-d]pyrimidin-4-yl)-1H-pyrazol-1-yl)-3-((cis)-3-methylcyclobutyl)propanenitrile, or a pharmaceutically acceptable salt thereof.

11. The phosphoric acid salt of the compound of claim 1.

12. A pharmaceutical composition comprising the compound of claim 1, or a pharmaceutically acceptable salt thereof, and at least one pharmaceutically acceptable carrier.

13. (canceled)

14. (canceled)

15. A method of treating a myeloproliferative disorder in a patient comprising administering to said patient a therapeutically effective amount of a compound of claim 1, or a pharmaceutically acceptable salt thereof.

16-18. (canceled)

19. The phosphoric acid salt of the compound of claim 4.

20. A pharmaceutical composition comprising the compound of claim 4, or a pharmaceutically acceptable salt thereof, and at least one pharmaceutically acceptable carrier.

21. A method of treating a myeloproliferative disorder in a patient comprising administering to said patient a therapeutically effective amount of a compound of claim 4, or a pharmaceutically acceptable salt thereof.
